(19) United States
(12) Patent Application Publication
(10) Pub. No.: US 2006/0069272 A1
(43) Pub. Date: Mar. 30, 2006

(54) Process for preparing 2,5-disubstituted 3-alkylthiophenes

(76) Inventors: Giorgio Bertolini, Milano (IT); Marco Frigerio, Milano (IT)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/530,858

(22) PCT Filed: Oct. 13, 2003
(86) PCT No.: PCT/IB03/04498

§ 371 Date: Apr. 8, 2005

(30) Foreign Application Priority Data
Oct. 14, 2002 (IT) MI2002A 002172

(51) Int. Cl. C07D 333/16

(52) U.S. Cl. 549/78

(57) ABSTRACT

The present invention relates to a process for preparing 2,5-disubstituted 3-alkyl-thiophenes and more particularly to a process for preparing them that comprises an acylation reaction in position 5 of 2-substituted 3-alkylthiophenes. This process does not need reagents which are difficult to handle and does not need anhydrous conditions or inert atmosphere. The resulting product is obtained in high purity.

The present invention relates to a novel process for preparing 2,5-disubstituted 3-alkyl-thiophenes and more particularly to a process for preparing them that comprises an acylation reaction in position 5 of 2-substituted 3-alkylthiophenes.

### PRIOR ART

2,5-Disubstituted 3-alkylthiophenes, of general formula 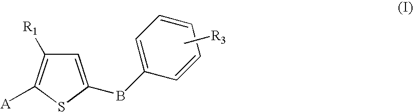
and in particular 2,3-dimethyl-5-benzylthiophene of formula 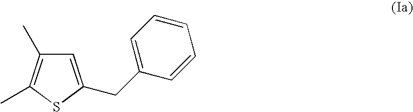
(R₁═A═CH₃, B═CH₂, R₃═H), are useful synthetic intermediates.

For example, in U.S. Pat. Nos. 6,121,271 and 6,103,708 and in international patent applications WO 99/61435 and WO 99/58522, compound (Ia) is used as an intermediate in the preparation of medicinal products for treating metabolic disorders related to insulin resistance or hyperglycemia.

Two different processes for preparing compound (Ia) are also described in the above-mentioned documents.

More particularly, international patent application WO 99/61435 illustrates the synthesis of compound (Ia) according to the sequence given below:

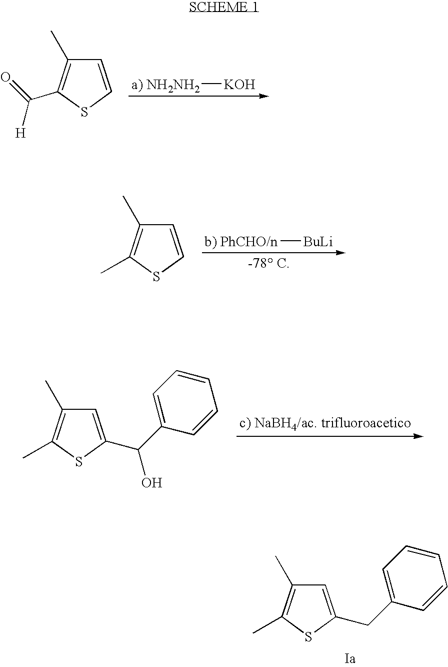

As may be seen, this synthesis involves the reduction of 3-methylthiophene-2-carboxaldehyde (step a) to give 2,3-dimethylthiophene, its functionalization in position 5 (step b) and, finally, subsequent reduction of the intermediate alcohol (step c) to give the desired compound (Ia).

However, this process is difficult to apply industrially since it involves the use of n-BuLi, which is a notoriously flammable reagent that requires an inert atmosphere, an anhydrous medium and a low temperature (−78° C.).

Another non-neglible drawback is represented by the need to use, in the condensation with benzaldehyde (step b), an intermediate 2,3-dimethylthiophene of high purity in order to minimize the possible side reactions and thus to obtain the desired product (Ia) in acceptable overall yields, which in this case would be about 67%, and with a sufficient degree of purity for subsequent direct synthetic use. The starting material for the process, i.e. 3-methylthiophene-2-carboxaldehyde, is, however, commercially available with a relatively poor degree of purity, generally of about 90%, and consequently the impure 2,3-dimethylthiophene obtained by reducing it cannot be used in unpurified form in the subsequent step, but rather is always subjected to purification; in particular, being a an oil, it is purified by chromatography, which is definitely not very method practicable from an industrial point of view.

Similarly, the final compound (Ia), which is a high-boiling liquid, is difficult to isolate and therefore another possible purification at level, by means of chromatography or distillation, would be relatively problematic on an industrial scale.

The second process for preparing compound (Ia), described in patents U.S. Pat. No. 6,121,271 and U.S. Pat. No. 6,103,708 and in international patent application WO 99/58522, may be represented according to the following scheme:

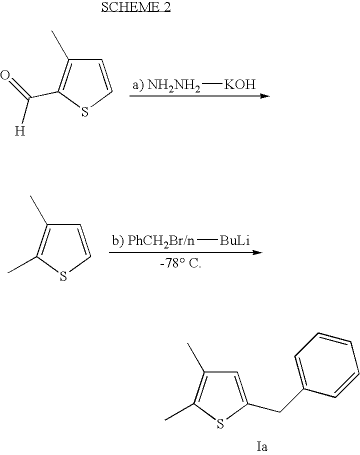

In this case also, the same drawbacks described above may be encountered, i.e. the use of n-BuLi, which is difficult to work with industrially, and the use of two successive chromatographies for the purification of the intermediate 2,3-dimethylthiophene and the final product (Ia), which are both liquids at room temperature.

We have now found a novel process for preparing 2,5-disubstituted 3-alkylthiophenes, in particular 2,3-dimethyl-5-benzylthiophene, which is significantly advantageous in industrial terms since it does not use reagents that are relatively difficult to work with such as n-BuLi, it does not require anhydrous conditions or inert atmospheres, and it appreciably simplifies the purification stages. This process makes it possible above all to use commercially available technical-grade starting materials directly, and to obtain the final product (I) in yields comparable to those for the known preparations but with high purity, entirely avoiding the use of chromatographic methods or distillation techniques, both on the intermediates and on the final product. The reason for this is that these final products generally have a purity that allows them to be used directly in subsequent processes without further purification.

### SUMMARY OF THE INVENTION

One subject of the present invention is therefore a process for preparing 2,5-disubstituted 3-alkylthiophenes of formula 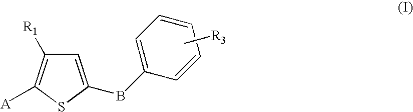
in which
A represents a CH₃, R₂CH₂, HOCH₂ or R₂CH(OH)— group,
B represents a CHOH or CH₂ group,
R₁ represents H or a C₁-C₅ alkyl group,
R₂ represents a C₁-C₅ alkyl group,
R₃ represents H or a C₁-C₅ alkyl group or a C₁-C₅ haloalkyl group, preferably CF₃, or a halogen chosen from fluorine, chlorine and bromine,
   which comprises:
   (a) the reaction of a compound of formula 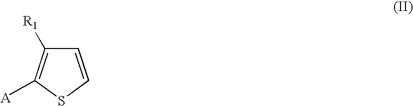
      in which
      A represents a CHO, CH₃, R₂CH₂ or R₂—CO— group, and
      R₁ and R₂ have the meanings given above;
      with a compound of formula 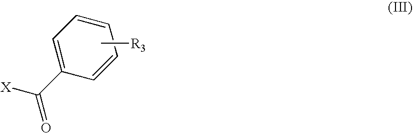
         in which
      X represents OH, halogen or a group of formula 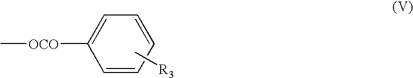
      or a group of formula
         —OCOOR₄ (VI)
         in which
      R₄ represents a C₁-C₅ alkyl, an optionally substituted benzyl or an optionally substituted aryl, and
      R₃ has the meanings given above;
      to give a compound of formula 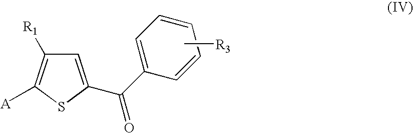
         in which
      A, R₁, R₂ and R₃ have the meanings given above; and
   (b) the reduction of the compound of formula (IV) thus obtained to give the compound of formula (I).

### DESCRIPTION OF THE INVENTION

The process that is the subject of the present invention comprises the acylation reaction (a) of the compound of formula II and the subsequent reduction (b) of the acyl intermediate of formula IV to give the desired compound I.

Generally, the starting compound of formula II is commercially available or may be prepared according to processes known to those skilled in the art. For example, in the case where it is desired to use the preferred compound IIb (R₁═A═CH₃) as starting compound, it may be prepared, in accordance with that described in the abovementioned prior art, by reduction, with hydrazine and potassium hydroxide, of the commercially available compound IIa (R₁═CH₃, A═CHO). As disclosed previously, an appreciable advantage of the present process is the direct use of the commercial compound IIa (purity of about 90%) in the reduction reaction to give IIb or, alternatively, in the acylation reaction (a) to give compound IVa (R₁═CH₃, A═CHO, R₃═H).

The acylation reaction referred to in point (a) is generally performed under the standard conditions of Friedel-Crafts acylations.

The acylating compound of formula III may be a carboxylic acid (X═OH), an acid halide (X=halogen), preferably chloride (X═Cl), a symmetrical anhydride (X═V) or a mixed anhydride (X═VI).

Preferred mixed anhydrides are those in which R₄ represents methyl, ethyl, i-butyl or benzyl, more preferably methyl or ethyl.

Acyl halides are preferred acylating agents, and benzoyl chloride is even more preferred (IIIa, X═Cl, R₃═H).

The catalysts that may be used in this reaction are generally Lewis acids, for instance AlCl₃, SbF₅, FeCl₃, TiCl₄ or BF₃, preferably AlCl₃.

Suitable solvents are those commonly used by those skilled in the art for reactions of this type, i.e. chlorinated solvents such as methylene chloride, chloroform or trichloroethane, or deactivated aromatic solvents such as nitrobenzene, preferably methylene chloride.

In the acylation reaction (a), a molar ratio of compound III/Lewis acid/compound II generally of between 0.9-1.5/0.9-1.5/1 and preferably of about 1/1/1 per unit of compound II is used.

The second step of the present process (step b) involves the reduction of the intermediate of formula IV to give the desired product of formula I.

Depending on the structure of the intermediate IV and of the final compound I, this reduction may be performed by means of a single reductive treatment (b) or, preferably, by means of a first reduction reaction (b₁), optionally followed by a second reduction reaction (b₂).

In the case where it is desired to obtain the completely reduced compounds of formula I directly, a single reductive treatment of the compound of formula IV will be performed to give the compound of formula I in which A═CH₃ or R₂CH₂ and B═CH₂.

In this case, a reduction reaction may be performed under the standard conditions for reducing C═O groups to CH₂ groups, for example by using a borohydride in the presence of a strong acid, for instance trifluoroacetic acid, methanesulphonic acid or sulphuric acid, preferably sodium borohydride or sodium cyanoborohydride in the presence of trifluoroacetic acid, or zinc iodide or trimethylsilyl chloride or trialkylsilanes in the presence of trifluoroacetic acid.

Alternatively, the process may be performed by means of a first reduction reaction (b₁) of the compound of formula 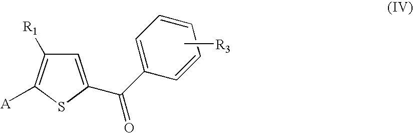
in which A represents a CHO, CH₃, R₂CH₂ or R₂—CO— group, to give the hydroxylated intermediate compound of formula 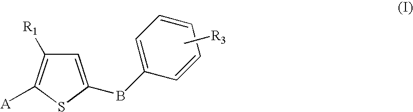
in which A represents a CH₃, R₂CH₂, HOCH₂ or R₂CH(OH)— group and B═CHOH, optionally followed by a second reduction reaction (b₂) of the hydroxylated intermediate of formula I as defined above, to give the final compound of formula 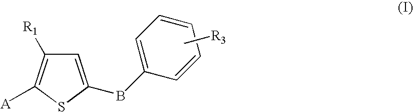
in which A represents a CH₃ or R₂CH₂ group and B═CH₂.

In the first step (b₁), suitable conditions may be used to perform the reduction of the C═O groups to CHOH groups, for instance treatment with metal hydrides such as sodium borohydride, lithium aluminium hydride or boranes, preferably with sodium borohydride in alkaline medium, or alternatively by treatment with aluminium isopropoxide (Meerwein-Ponndorf-Verley reaction).

The intermediate hydroxylated derivatives of formula I as defined above, preferably the compounds Ib (R₁═CH₃, A═CH₂OH, B═CHOH, R₃═H) or Ic (R₁═A═CH₃, B═CHOH, R₃═H) may then be subjected to the subsequent reduction step (b₂), directly or, preferably, after purification.

This purification is advantageously performed by crystallization, since the said hydroxylated intermediates are generally crystalline solids.

Suitable crystallization solvents are usually apolar solvents such as ethers, for example diethyl ether, diisopropyl ether or methyl tert-butyl ether, aromatic hydrocarbons, for example toluene or xylene, aliphatic hydrocarbons, for example hexane, heptane or cyclohexane, or mixtures of these solvents.

By then performing this preferred variant of the process, which involves a first reduction (CO->CHOH, b₁), the crystallization of the hydroxylated intermediate and its subsequent total reduction (CHOH->CH₂ b₂), it is thus possible to perform the entire synthetic sequence of the present invention without the need for chromatographic purifications or distillations, which are drawbacks from an industrial point of view.

The hydroxylated compounds of formula I, preferably the abovementioned compounds Ib and Ic, which are preferably purified, may then be converted into the corresponding completely reduced compounds of formula I, preferably into compound Ia, by reduction (b₂) of the CHOH groups to CH₂ groups, for example by treatment with a borohydride in the presence of a strong acid, for instance trifluoroacetic acid, methanesulphonic acid or sulphuric acid, preferably with sodium borohydride or sodium cyanoborohydride and trifluoroacetic acid, or zinc iodide, or, surprisingly, by means of catalytic hydrogenation.

This latter reaction may be performed simply by treating the hydroxylated compound I, dissolved in a suitable solvent such as an alcohol, for instance methanol, ethanol or isopropanol, preferably methanol, or in mixtures of water and alcohols at a hydrogen pressure of between about 1 and 10 bar, at a temperature of between about 15 and 60° C., in the presence of a hydrogenation catalyst chosen from palladium and platinum, preferably palladium supported on an inert support such as carbon, alumina, silica or zeolites, preferably on carbon, in a neutral or acidic medium, without observing the foreseeable poisoning of the catalyst normally caused by sulphur-containing compounds, as are the thiophene derivatives prepared in the present process.

The general scheme of the process that is the subject of the present invention is given hereinbelow for greater clarity:

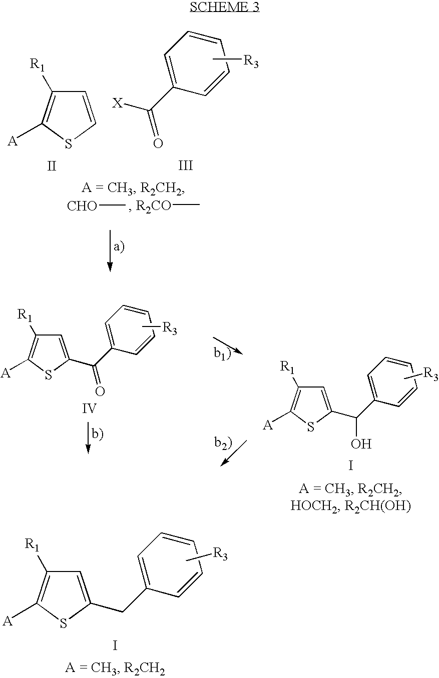

The possible variants of the reduction step (b, b₁, b₂), which are within the scope of the process according to the present invention, are shown in this scheme. The choice of the optimum sequence and of the most advantageous reduction conditions forms part of the normal capabilities of a person skilled in the art.

One preferred embodiment of the present invention is represented by the preparation of 2,3-dimethyl-5-benzylthiophene (Ia) according to the following scheme:

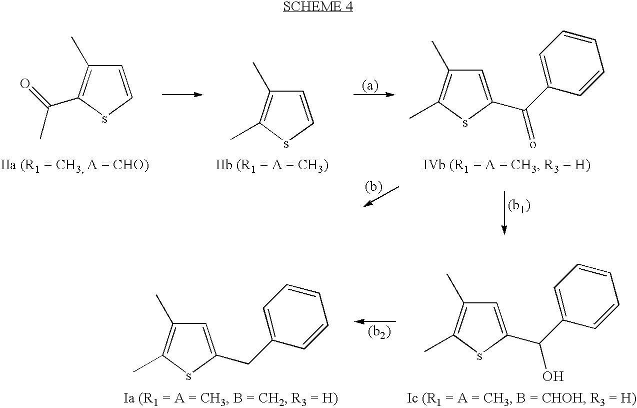

In particular, the product IIb may be prepared from the commercially available low-quality compound IIa, by reduction with hydrazine and sodium hydroxide, as described, for example, in WO 99/61435, and used in the next step without being isolated or purified.

Specifically, the acylation reaction (a) of compound IIb, preferably performed with benzoyl chloride and AlCl₃, may be performed by directly using its organic extraction solution, which is preferably methylenic, originating from the reduction of compound IIa.

Compound IVb thus obtained may be totally reduced (b), to give Ia, for example by reaction with sodium borohydride and trifluoroacetic acid or, preferably, subjected to two successive reduction reactions (b₁ and b₂) with formation of the hydroxylated intermediate Ic. This latter variant of the process is particularly preferred, since it allows the intermediate Ic to be purified by crystallization.

In particular, the first reduction reaction (b₁) is preferably performed with sodium borohydride and sodium hydroxide, while the second reduction with sodium borohydride and trifluoroacetic acid or by means of catalytic hydrogenation, preferably with sodium borohydride and trifluoroacetic acid. The intermediate Ic is generally crystallized from apolar solvents such as ethers, for example diethyl ether, diisopropyl ether or methyl tert-butyl ether, aromatic hydrocarbons, for example toluene or xylene, or aliphatic hydrocarbons, for example hexane, heptane or cyclohexane, or mixtures of these solvents, preferably from n-heptane.

The preferred process given in Scheme 4, in particular in its preferred variant, comprising the two successive reduction reactions b₁ and b₂ and the crystallization of the hydroxylated intermediate Ic makes it possible to prepare compound Ia in an overall yield that is comparable with that obtained according to the abovementioned known processes, but avoiding the reagents, reaction conditions and purification techniques of the prior art that are difficult to apply industrially.

In addition, as mentioned previously, the present process may be applied using the commercially available low-quality product IIa. Specifically, by using compound IIa with a GC purity of 90%, it is possible to obtain 2,3-dimethyl-5-benzylthiophene Ia with an HPLC purity of about 99%, without the need for chromatographic purifications, but merely by means of a simple crystallization of the intermediate alcohol Ic.

Finally, another preferred embodiment of the present invention is represented by the preparation of compound I according to the following scheme:

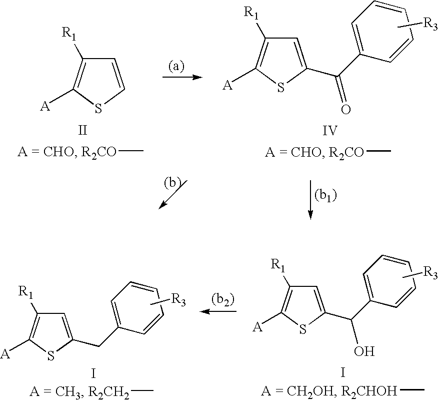

In this case, the product II, in which A represents CHO or R₂CO—, is acylated directly to give the intermediate IV which is then subjected to the simultaneous reduction of both the carbonyl groups.

By analogy with that described previously, this conversion may be performed by means of a single reduction reaction (b) or, preferably, two successive reduction reactions (b₁ and b₂), with the possibility of purification of the intermediate hydroxylated compound by crystallization.

One particularly preferred embodiment of this process is the preparation of 2,3-di-methyl-5-benzylthiophene (Ia), from 3-methyl-2-thiophenecarboxaldehyde (IIa, A═CHO, R₁═CH₃) by acylation (a) with benzoyl chloride, reduction (b₁) of compound IVa (A═CHO, R₁═CH₃, R₃═H) to the diol Ib (A═CH₂OH, R₁═CH₃, R₃═H), purification thereof by crystallization and subsequent reduction (b₂) to give the final compound Ia (A═R₁═CH₃, R₃═H).

The examples that follow will now be given for the purpose of illustrating the present invention more clearly.

### EXPERIMENTAL SECTION

### Example 1

### Preparation of 2,3-dimethylthiophene (IIb, R₁═A═CH₃)

3-Methyl-2-thiophenecarboxaldehyde (Aldrich, 90% GC purity) (40 g) was dissolved in diethylene glycol (144 ml) and hydrazine hydrate (62 ml) was added slowly. The reaction mixture was refluxed (about 126° C.) for 30 minutes. The solution was cooled to 30-40° C. and potassium hydroxide (46 g) was then added portionwise. The solution was then slowly heated to reflux and stirred at this temperature for one and a half hours.

The reaction mixture was cooled to room temperature and poured into cold water (1 L), acidified to pH 2 with concentrated hydrochloric acid (about 150 ml) and extracted with methylene chloride (2×100 ml). The combined organic phases were dried over anhydrous sodium sulphate. The solid was filtered off and washed with methylene chloride (20 ml) and the 2,3-dimethylthiophene solution was used directly without further purification (88.2% GC purity).

### Example 2

### Preparation of 2,3-dimethyl-5-benzoylthiophene (IVb, R₁═A═CH₃, R₃═H)

Benzoyl chloride (37.2 ml) was dissolved in the methylenic solution containing about 35.6 g of 2,3-dimethylthiophene obtained from Example 1. This solution was added slowly at 0-5° C. to a solution of anhydrous aluminium trichloride (42.7 g) in methylene chloride (100 ml) over 2-3 hours. The reaction mixture was stirred at 20-25° C. for 2 hours and was then refluxed for one hour. After cooling to room temperature, the reaction mixture was poured Into water (500 ml) at 0° C. and stirred for 15 minutes. The two phases were separated and the organic phase was washed first with water (500 ml) and then with aqueous 30% sodium hydroxide solution (30 ml). The organic phase was evaporated under vacuum to give 64.5 g of 2,3-dimethyl-5-benzoylthiophene (94% yield over two steps) (88.8% GC purity).

¹H-NMR (300 MHz, DMSO) δ: 7.78 (d, 2H), 7.64 (t, 1H), 7.54 (t, 2H), 7.43 (s, 1H), 2.39 (s, 3H), 2.12 (s, 3H).

### Example 3

### Preparation of 2,3-dimethyl-5-(α-hydroxybenzyl)thiophene (Ic, R₁═A═CH₃, B═CHOH, R₃═H)

Sodium borohydride (7.0 g) was dissolved in water (40 ml) and 30% sodium hydroxide (2 ml) and this solution was added slowly over about one hour to a solution of 2,3-dimethyl-5-benzoylthiophene (64.5 g) in methanol (250 ml) and 30% sodium hydroxide (3.5 ml). The solution was heated at 35° C. for 6 hours and then refluxed for 2 hours. The reaction mixture was then cooled to 35-40° C. and the methanol was evaporated off under vacuum. The residue was dissolved in water (150 ml), 30% sodium hydroxide (20 ml) and methylene chloride (150 ml). The two phases were separated and the aqueous phase was extracted with methylene chloride (50 ml). The organic phase was evaporated under vacuum and the crude product was dissolved in hot n-heptane (700 ml) and crystallized to give, after drying at 40° C., 46.0 g of 2,3-dimethyl-5-(α-hydroxybenzyl)thiophene (71% yield) (99.1% GC purity).

m.p.=79-80° C., ¹H-NMR (300 MHz, DMSO) δ: 7.40-7.23 (m, 5H), 6.52 (s, 1H), 6.03 (s, 1H), 5.72 (s, 1H), 2.21 (s, 3H), 1.99 (s, 3H).

### Example 4

### Preparation of 2,3-dimethyl-5-benzylthiophene Ia, R₁═A═CH₃, B═CH₂, R₃═H)

2,3-Dimethyl-5-(α-hydroxybenzyl)thiophene (46.0 g) was dissolved in THF (1100 ml), followed by addition of sodium borohydride (23.9 g). The reaction mixture was cooled to 0° C. and trifluoroacetic acid was added slowly over about two hours at 0-5° C. The reaction mixture was stirred at 0-5° C. for 2 hours. A 30% solution of sodium hydroxide (150 ml) in water (300 ml) was added and the two phases were separated.

The organic phase was evaporated under vacuum and the residue was dissolved in methylene chloride. The salts were filtered off and the organic solution was washed with aqueous sodium chloride solution and evaporated under vacuum to give 40.5 g of 2,3-dimethyl-5-benzylthiophene as an oil (95% yield) (98.8% HPLC purity).

¹H-NMR (300 MHz, DMSO) δ: 7.32-7.20 (m, 5H), 6.54 (s, 1H), 3.99 (s, 2H), 2.21 (s, 3H), 2.02 (s, 3H).

### Example 5

### Preparation of 2,3-dimethyl-5-benzylthiophene Ia, R₁═A═CH₃, B═CH₂, R₃═H)

2,3-Dimethyl-5-(α-hydroxybenzyl)thiophene (46.0 g) was dissolved in methanol (750 ml), followed by addition of 5% palladium-on-charcoal containing 50% water (7.5 g on a dry basis). The reaction mixture was hydrogenated at 40-45° C. and 6-7 bar for 24 hours, to give 26 g of 2,3-dimethyl-5-thiophene (61% yield).

1. Process for preparing 2,5-disubstituted 3-alkylthiophenes of formula 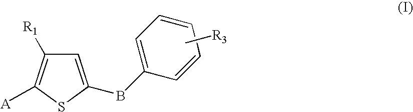
in which
A represents a CH₃, R₂CH₂, HOCH₂ or R₂CH(OH)— group,
B represents a CHOH or CH₂ group,
R₁ represents H or a C₁-C₅ alkyl group,
R₂ represents a C₁-C₅ alkyl group,
R₃ represents H or a C₁-C₅ alkyl group or a C₁-C₅ haloalkyl group, or a halogen chosen from fluorine, chlorine and bromine,
which comprises:
(a) reaction of a compound of formula 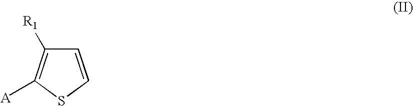
in which
A represents a CHO, CH₃, R₂CH₂ or R₂—CO— group, and
R₁ and R₂ have the meanings given above;
with a compound of formula 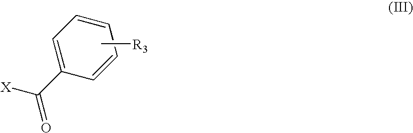
in which
X represents OH, halogen or a group of formula 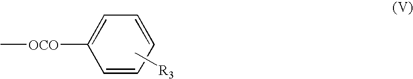
or a group of formula
—OCOOR₄ (VI)
in which
R₄ represents a C₁-C₅ alkyl, an optionally substituted benzyl or an option-ally substituted aryl, and
R₃ has the meanings given above;
to give a compound of formula 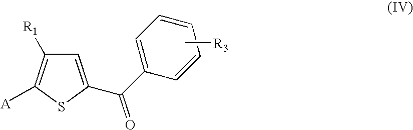
in which
A, R₁, R₂ and R₃ have the meanings given above; and
(b) reduction of the compound of formula IV thus obtained to give the compound of formula I.

2. The process of claim 1 for the preparation of 2,5-disubstituted 3-alkylthio-phenes of formula 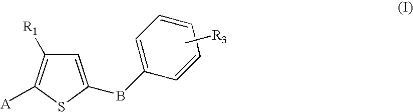
in which
A represents a CH₃ or R₂CH₂ group,
which comprises:
(a) reaction of a compound of formula 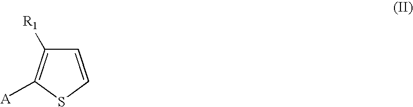
in which
A represents a CH₃ or R₂CH₂ group,
with a compound of formula 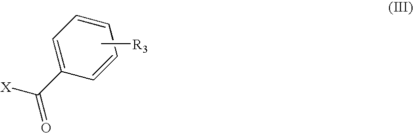
to give a compound of formula 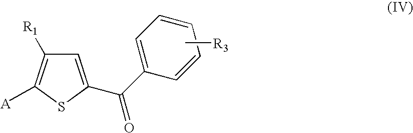
in which
A represents a CH₃ or R₂CH₂ group, and
(b) reduction of the compound of formula IV thus obtained to give the compound of formula I.

3. The process of claim 1 for the preparation of 2,5-disubstituted 3-alkylthiophenes of formula 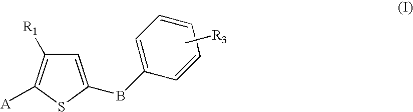
in which
A represents a CH₃, R₂CH₂, HOCH₂ or R₂CH(OH)— group,
which comprises:
(a) reaction of a compound of formula 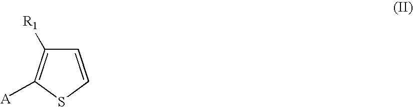
in which
A represents a CHO or R₂—CO— group,
with a compound of formula 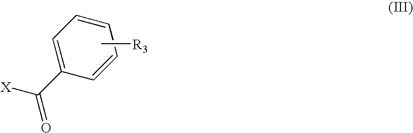
to give a compound of formula 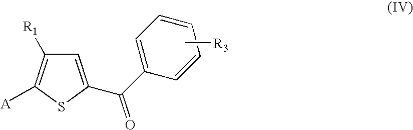
in which
A represents a CHO or R₂—CO group, and
(b) reduction of the compound of formula IV thus obtained to give the compound of formula I.

4. The process of claim 1 in which the reaction mentioned in point (a) is performed with a compound of formula III, in which X represents halogen, in the presence of a Lewis acid and in a solvent chosen from chlorinated solvents and deactivated aromatic solvents in the presence of AlCl₃ and in methylene chloride, in which the molar ratio of compound III/Lewis acid/compound II is between 0.9-1.5/0.9-1.5/1.

5. Process according to claim 1, in which the reduction mentioned in point (b) is performed by means of a single reductive treatment of the compound of formula 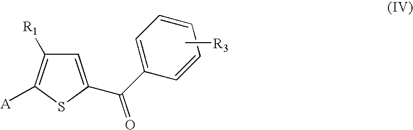
to give the compound of formula 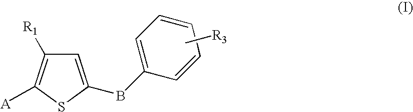
in which
A represents a CH₃ or R₂CH₂ group, and
B represents a CH₂ group.

6. Process according to claim 5, in which the said reductive treatment is performed with sodium borohydride or sodium cyanoborohydride in the presence of trifluoro-acetic acid.

7. Process according to claim 1, in which the reduction mentioned in point (b) is performed by means of a first reduction reaction (b₁) of the compound of formula 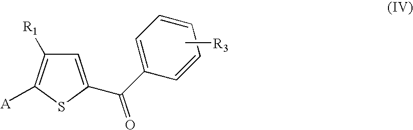
to give the hydroxylated intermediate compound of formula 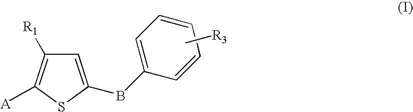
in which
A represents a CH₃, R₂CH₂, HOCH₂ or R₂CH(OH)— group, and
B represents a CHOH group;
optionally followed by a second reduction reaction (b₂) of the said hydroxylated intermediate of formula I to give a final compound of formula 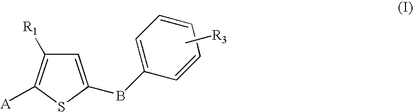
in which
A represents a CH₃ or R₂CH₂ group, and
B represents a CH₂ group.

8. Process according to claim 7, in which the reduction mentioned in point (b) is performed by means of the said reduction reactions (b₁) and (b₂) successively.

9. The process according to claim 7, in which the first of the said reduction reactions (b₁) is performed by treatment with metal hydrides selected from the group consisting of sodium borohydride, lithium aluminium hydride, boranes, and mixtures thereof, or by treatment with aluminium isopro-poxide.

10. The process of claim 7, in which the second of the said reduction reactions (b₂) is performed by treatment with a borohydride in the presence of a strong acid selected from the group consisting of trifluoroacetic acid, methanesulphonic acid, sulphuric acid, and mixtures thereof, or with zinc iodide or by catalytic hydrogenation.

11. The process of claim 7, in which the second of the said reduction reactions (b₂) is performed by catalytic hydrogenation of the hydroxylated intermediate of formula I (B═CHOH) dissolved in a suitable solvent at a hydrogen pressure of between 1 and 10 bar, at a temperature of between 15 and 60° C., in the presence of a hydrogenation catalyst chosen from palladium and platinum, supported on an inert support in a neutral or acidic medium.

12. Process according to claim 7, in which the said hydroxylated intermediate compound of formula 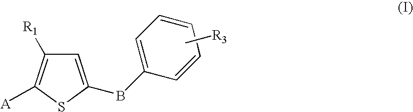
in which
A represents a CH₃, R₂CH₂, HOCH₂ or R₂CH(OH)— group, and
B represents a CHOH group
is purified by crystallization.

13. Process for preparing 2,3-dimethyl-5-benzylthiophene of formula 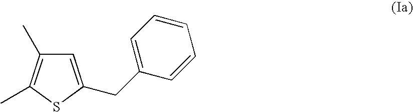
which comprises:
(a) the reaction of the compound of formula 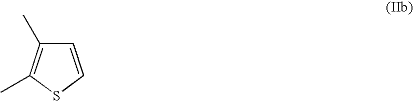
with a compound of formula 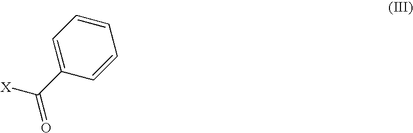
in which X represents halogen;
to give the compound of formula 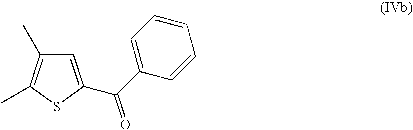
(b₁) the reduction of compound IVb to give the hydroxylated intermediate compound of formula 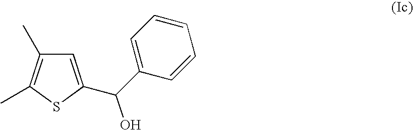
and
(b₂) the reduction of the hydroxylated intermediate compound Ic to give 2,3-dimethyl-5-benzylthiophene (Ia).

14. The process of claim 13, in which the reaction (a) is performed in the presence of a Lewis acid and a molar ratio of compound III/Lewis acid/compound II ranges from 0.9-1.5/0.9-1.5/1, the reduction reaction (b₁) is preformed by treatment wit a metal hydride, and the reduction reaction (b₂) is performed by treatment with a borohydride in the presence of a strong acid selected from the group consisting of trifluoroacetic acid, methanesulfonic acid, or sulfuric acid, or in the presence of zinc iodide or by catalytic hydrogenation.

15. The process of claim 13, in which the hydroxylated intermediate compound of formula Ic is purified by crystallization.

16. The process of claim 4, wherein the solvent is benzoyl chloride.

17. The process of claim 4, wherein the molar ratio of compound III/Lewis acid/compound II is 1/1/1.

18. The process of claim 7, wherein the second reduction reaction (b₂) is performed by treatment with sodium borohydride.

19. The process of claim 7, wherein the second reduction reaction (b₂) is performed by treatment with sodium borohydride and trifluoroacetic acid.

20. The process of claim 7, wherein the hydrogenation catalyst is palladium supported on an inert support.

21. The process of claim 7, wherein the inert support is selected from the group consisting of carbon, alumina, silica, zeolite, and mixtures thereof.

22. The process of claim 7, wherein the inert support is carbon.

23. The process of claim 13, wherein the hydroxylated intermediate compound of formula 1c is purified by crystallization from n-heptane.

24. The process of claim 11, wherein the suitable solvent is an alcohol or a mixture of water and the alcohol.

25. The process of claim 11, wherein the alcohol is selected from the group consisting of methanol, ethanol, isopropanol, and mixtures thereof.

26. The process of claim 11, wherein the alcohol is methanol.

26. The process of claim 11, wherein the hydrogenation catalyst is palladium.
